(19) United States
(12) Patent Application Publication
(10) Pub. No.: US 2026/0232181 A1
(43) Pub. Date: Aug. 13, 2026

(54) PASSIVE EYE CONDITION DETECTION DURING INDEPENDENT CONTENT PRESENTATION

(71) Applicant: Heru Inc., Miami, FL (US)

(72) Inventor: Mohamed ABOU SHOUSHA, Fort Lauderdale, FL (US)

(73) Assignee: Heru Inc., Miami, FL (US)

(21) Appl. No.: 19/048,832

(22) Filed: Feb. 7, 2025

(51) Int. Cl. A61B 3/00; A61B 3/024; G16H 50/20

(52) U.S. Cl. CPC A61B 3/005; A61B 3/024; G16H 50/20; A61B 2560/0462

(57) ABSTRACT

In some embodiments, a head-mounted device and related method may use a presenting application to present content to an eye while a testing application sandboxed from the presenting application generates one or more eye condition indications. Some embodiments may do so while preserving computer resources by using a hierarchical arrangement of machine learning models.

### SUMMARY

Eye condition testing technology combines healthcare diagnostics with computing systems to detect and monitor vision disorders and eye-related diseases. Changes in eye structure, responses to stimuli, and other eye-related data may indicate good eye health or one or more types of eye-related disorders or more systemic disorders. Early detection of these conditions may reveal indicators of broader health issues, such as hypertension and diabetes. However, many potential patients do not have the resources or initiative to visit a clinic or other healthcare setting to check their vision until significant problems have already arisen.

Conventional eye testing hardware and software face several practical considerations in clinical settings. Traditional eye exams require in-person visits with specialized equipment, while many vision tests rely on patient feedback through comparative choices, which can introduce variability in results due to a patient's frequent failures to fully comply with testing instructions. Testing equipment and software typically represent significant investments in skill and time for practices. Even when vision testing is performed with a head-mounted device, the brief duration of eye testing procedures may result in erroneous or missed diagnosis.

Some embodiments may resolve such issues or other issues by passively detecting eye conditions using a separate application to monitor eye data while a user is engaging with content presented on one or more other applications. In connection with a first application presenting image content to a user via an inward-facing display of a head-mounted device, some embodiments may obtain feedback data by measuring reflections off the user's eye using one or more sensors of the head-mounted device. For example, while a user is watching a video on a virtual reality headset, sensors inside the headset capture how the user's eyes react to the video. This feedback data is then provided to a second application that is sandboxed with respect to the first application. For example, eye reaction data (e.g., feedback data collected by an infrared sensor) for an eye watching a videogame cutscene may be sent to a separate health monitoring application that runs independently from the gaming application. The health monitoring application may be passively executing concurrently with the gaming application without interrupting the operations of the gaming application. The second application may then generate an eye condition indication by using a machine learning model based on the feedback data. For example, a health monitoring application may analyze eye reaction data using a neural network to determine if the user has any potential eye conditions.

By using an application to passively monitor eye health while image content is being presented to a user, some embodiments may collect significantly greater amounts of data to generate results with far greater accuracy. Additionally, or alternatively, such data collection may be performed without negatively impacting the user experience related to eye health detection. For example, such data collection may be performed alongside applications that a user is already engaging with on a regular basis (e.g., performed as background processes or without interrupting the user's current experience with an independent application with which the users are already engaging). Thus, for example, such embodiments may avoid or reduce the need to subject the user to visual testing content separate from content with which the user is engaging via an independent application). Furthermore, this concurrent execution allows for eye health detection to be accessible to a wider audience. Additionally, or alternatively, in cases where a content-presenting application may be causing eye damage, some embodiments may use a concurrently executing eye testing application to detect symptoms of such damage and stop or change operations of the content-presenting application.

Furthermore, the use of VR headsets or other head-mounted devices present a significant opportunity to address this need, by permitting the passive examination of eye conditions during use of the head-mounted device for unrelated entertainment or professional purposes. However, head-mounted devices often have limited computing resources, which may restrict their ability to perform intensive machine learning tasks, such as eye health diagnosis. The limited computing resources on these devices, such as CPU, GPU, and memory capacity, often require a careful balancing of application operations to prevent offloading tasks to external servers, which may introduce latency and reduce real-time performance. Additionally, the high-power consumption of machine learning operations can quickly drain the battery of VR headsets or other mobile, head-mounted devices, limiting their usage duration. This situation may be exacerbated if the head-mounted device is being used to perform additional operations during testing procedures.

Some embodiments may resolve such issues and other issues by detecting eye conditions, while employing a layered approach with multiple machine learning models. Some embodiments may obtain a feedback data stream from a set of inward-facing sensors of a head-mounted device while presenting image content through its inward-facing display. For example, while a user is watching a video on a head-mounted device, the head-mounted device may use inward-facing sensors to collect a feedback data stream. Some embodiments may then determine a first prediction model output of a first prediction model by providing the feedback data stream to the first prediction model. In response to the prediction model output satisfying a set of criteria, some embodiments may generate an eye condition indication, by providing the feedback data stream as inputs to a second prediction model that outputs the eye condition indication. The second prediction model may also receive, as additional input, historical data received in the collection period from before the second prediction model was first triggered to generate the eye condition indication.

By employing a layered approach with multiple machine learning models, some embodiments may efficiently detect eye conditions while preserving computing resources. Moreover, using both historical and current data provides greater context, leading to more accurate eye condition diagnoses. By employing the system and operations described in this disclosure, some embodiments may possess an enhanced ability to detect subtle changes and trends in eye health.

Various other aspects, features, and advantages of the invention will be apparent through the detailed description of the invention and the drawings attached hereto. It is also to be understood that both the foregoing general description and the following detailed description are examples and are not restrictive of the scope of the invention.

### BRIEF DESCRIPTION OF THE DRAWINGS

FIG. 1 shows an example system for passively detecting eye conditions during content presentation, in accordance with one or more embodiments.

FIG. 2 is an example head-mounted device, in accordance with one or more embodiments.

FIG. 3 shows an example conceptual diagram of content presentation being processed for detecting eye conditions, in accordance with one or more embodiments.

FIG. 4 shows a flowchart of a process for detecting eye conditions during content presentation, in accordance with one or more embodiments.

FIG. 5 shows a flowchart of a process for detecting eye conditions by generating simulated input data based on feedback data, in accordance with one or more embodiments.

FIG. 6 shows an example conceptual architecture for preserving computer resources while detecting eye conditions, in accordance with one or more embodiments.

FIG. 7 shows a flowchart of a process for preserving computer resources, while detecting eye conditions, in accordance with one or more embodiments.

The technologies described herein will become more apparent to those skilled in the art by studying the detailed description in conjunction with the drawings. Embodiments of implementations describing aspects of the invention are illustrated by way of example, and the same references can indicate similar elements. While the drawings depict various implementations for the purpose of illustration, those skilled in the art will recognize that alternative implementations can be employed without departing from the principles of the present technologies. Accordingly, while specific implementations are shown in the drawings, the technology is amenable to various modifications.

### DETAILED DESCRIPTION OF THE DRAWINGS

In the following description, for the purposes of explanation, numerous specific details are set forth in order to provide a thorough understanding of the embodiments of the invention. It will be appreciated, however, by those having skill in the art that the embodiments of the invention may be practiced without these specific details or with an equivalent arrangement. In other cases, well-known structures and devices are shown in block diagram form in order to avoid unnecessarily obscuring the embodiments of the invention.

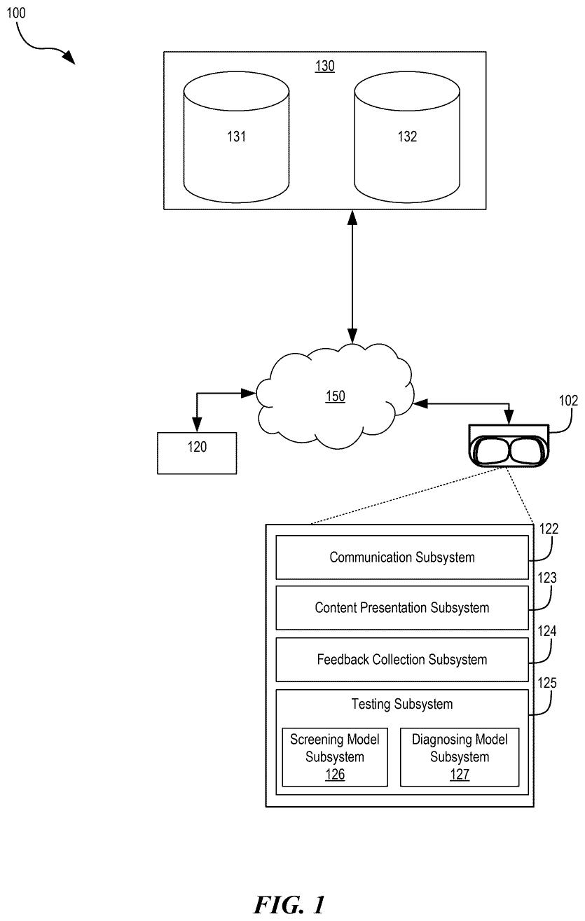

FIG. 1 shows an example system 100 generating a network command by using descriptors of attributes or payloads, in accordance with one or more embodiments. A system 100 includes a client device 102 in communication with a server 120 via a network 150. As will be described further in this disclosure, the client device 102 may perform operations to passively detect eye conditions during content presentation.

In some embodiments, the system 100 may passively detect eye conditions as unrelated or independent image content is presented on a head-mounted device. For example, in connection with a first application presenting image content to a user via an inward-facing display of a head-mounted device, some embodiments may obtain feedback data by measuring reflections off the user's eye using one or more sensors of the head-mounted device. For example, in connection with a presenting application displaying image content to a user via the inward-facing display of a head-mounted device, some embodiments may obtain eye-related feedback data through one or more inward-facing sensors. This feedback data may include data such as eye response data corresponding to the user's eye responses to the presented image content and reflected image content data corresponding to reflections off the user's eye while the image content is being displayed. The feedback data is provided to a second application that is sandboxed from the first application. This second application, such as a health monitoring application, may run independently and concurrently with the first application without interrupting its operations. The second application may then use a machine learning model to generate an eye condition indication based on the feedback data. By collecting this feedback data for use by a testing application while content is being provided by a separate presenting application, some embodiments may capture large volumes of eye-related data useful for detecting eye conditions with a lower risk of a user growing bored or frustrated with a test.

While a first application presents image content to a user via an inward-facing display of a head-mounted device, some embodiments may obtain feedback data using one or more sensors of the head-mounted device. Some embodiments may then provide this feedback data to a machine learning model of a second application. Some embodiments may then generate an eye condition indication by providing the feedback data to a machine learning model. For example, a health monitoring application may analyze eye reaction data using a deep neural network to determine if the user has any likely eye conditions and generate a corresponding eye condition indication. By using a local machine learning model of the second application to detect eye conditions, some embodiments may reduce network communication costs and increase the likelihood of obtaining an accurate detection of one or more eye conditions.

Some embodiments may use one or more machine learning models to generate sub-results that are then used to generate an eye condition indication. For example, some embodiments may use the second application to create simulated input data (e.g., a reconstructed image) using image processing algorithms based on the eye-related feedback data and then provide, as an input, this simulated input data in conjunction with the feedback data to a machine learning model. Alternatively, or additionally, some embodiments may use a machine learning model to generate simulated input data, use a machine learning model to predict an eye response (e.g., a gaze-change), and determine an eye condition indication based on differences between the predicted eye response and a measured response in the feedback data. By generating sub-results that are then used to assess health conditions, some embodiments may provide a means of explaining an eye-related condition or other condition, where such explainability may be useful or required in various clinical settings.

In some embodiments, the system 100 may passively detect eye conditions, as unrelated image content is presented to a head-mounted device. Some embodiments present image content to a user's eye via the inward-facing display of a first application and obtain a feedback data stream, comprising eye response data and reflection data, using one or more inward-facing sensors while the image content is being presented during a first period. Some embodiments use the feedback data stream to determine a prediction model output with a screening model and, upon the output triggering a set of criteria, generate an eye condition indication using a greater-complexity model with the feedback data stream or historical data.

Some embodiments may display image content to a user's eye through the inward-facing display of a first application and collect a feedback data stream, which includes eye response data and reflection data, using one or more inward-facing sensors while the image content is shown during a first period. Some embodiments may then determine a first prediction model output by providing the feedback data stream, as learning model inputs to a screening machine learning model. By using the feedback data stream collected from the user's eye responses and reflections to generate a first prediction model output, some embodiments may provide the benefit of identifying potential issues or conditions by analyzing the feedback data. Furthermore, such a model may gatekeep the use of more computationally intensive models. For example, based on the first prediction model output, some embodiments may provide the feedback data stream to a greater-complexity machine learning model.

Some embodiments present image content to a user's eye via an inward-facing display, obtain a feedback data stream using sensors, and determine a model-triggering prediction value with a screening machine learning model during a first period. Some embodiments may then determine whether the model-triggering prediction value satisfies a set of triggering criteria and, in response to the output satisfying the set of triggering criteria, initialize the use of a greater-complexity prediction model to determine eye conditions. Some embodiments generate an eye condition indication by providing, as a set of inputs, the feedback data stream and historical data obtained during the first period to a greater-complexity machine learning model during a second period. In some embodiments, the first application continues to present the image content.

The client device 102 may include a head-mounted device, such as a virtual reality device or an augmented glasses device. The client device 102 may send requests, responses, or other messages to the server 120 that may require communication with other computing devices or other electronic devices. Additionally, the server 120 may include various types of computing units, such as physically separate servers, virtual nodes hosted on one or more physical machines, or nodes on a cloud computing system. Applications, services, or other operations may use data provided by the client device 102, the server 120, or a set of databases 130 that includes a first networked database 131 and a second networked database 132. The set of databases 130 may include various types of databases, such as SQL databases, no SQL databases, graph databases, etc. In some embodiments, the client device 102 may perform one or more operations related to a communication subsystem 122, a content presentation subsystem 123, a feedback collection subsystem 124, or a testing subsystem 125, where the testing subsystem 125 may include a screening model subsystem 126 and a diagnosing model subsystem 127. The server 120 may perform operations such as training one or more machine learning operations described in this disclosure based on feedback data, generating machine learning models, storing feedback data, storing historical feedback data, etc.

In some embodiments, the communication subsystem 122 may obtain program instructions, commands, parameters, values, or other data from the server 120 or the set of databases 130. For example, the communication subsystem 122 may retrieve a set of parameters from the set of databases 130. Furthermore, operations performed by the client device 102 may use the communication subsystem 122 to send messages to the set of databases 130, the server 120, or another computing device described in this disclosure.

In some embodiments, a content presentation subsystem 123 may present image content to a user that is viewed by the user's eye. For example, a presenting application of the content presentation subsystem 123 may send rendered frames through the head-mounted device's runtime display, where the display may then warp content to match the head-mounted device's optical distortion profile. In some embodiments, the content presentation subsystem 123 may present interactive content.

In some embodiments, a feedback collection subsystem 124 may collect feedback data from the user's eyes. In some embodiments, the feedback collection subsystem 124 may collect feedback data from a set of inward-facing sensors, where the feedback data may then be presented to a sandboxed vision testing application that is sandboxed from one or more applications of the content presentation subsystem 123. For example, while a first application is presenting videogame or video content to a user via a head-mounted display, a set of inward-facing sensors may capture image data of a user's eyes, measure eye responses, or obtain other eye-related data and output feedback data that includes or is based on this data. As described elsewhere, the feedback collection subsystem 124 may then send this feedback data to the testing subsystem 125 (e.g., to the screening model subsystem 126 or the diagnosing model subsystem 127) for vision testing operations. Alternatively, or additionally, the output data may be provided back to the content presentation subsystem 123 for use to perform content-presenting operations, such as foveated rendering.

In some embodiments, a testing subsystem 125 may generate an eye condition indication based on the feedback data. In some embodiments, the testing subsystem 125 may use a machine learning model that receives feedback data as inputs and directly outputs eye condition indications. Various types of machine learning models or combinations of machine learning models may be used for this outputting eye condition indication, such as convolutional neural networks (CNNs), recurrent neural networks (RNNs), Long Short-Term Memory network (LSTM). For example, in some embodiments, the testing subsystem 125 may directly provide a sequence of images of an eye obtained by inward-facing cameras to a recurrent convolutional neural network (RCNN) to obtain labels process sequences of eye images, where the memory parameters of a machine learning models may be suited for tracking data across frames. Furthermore, a machine learning model may include multiple layers of different types of models, such as a first RCNN layer to track objects and generate labels, and a vision transformer to then generate an output eye condition indication.

Alternatively, or additionally, the testing subsystem 125 may use a machine learning model to perform sub-operations whose results are then used to contribute to determine a final diagnosis (e.g., by determining an eye condition indication). For example, some embodiments may generate a sequence of reconstructed images using a machine learning model based on available data, such as eye reflection data or head-mounted device utilization data. Various types of operations may be performed to reconstruct images, such as algorithmic approaches or machine learning model approaches. For example, some embodiments may use an algorithm approach, such as a gradient prior method that identifies and isolates reflection off an eye based on edge information captured in reflections of light off an eye, a non-negative matrix factorization (NMF) to separate an image of an eye into its constituent layers, and a deep image prior approach that exploits frequency changes between the different types of light from an eye. Alternatively, or additionally, the testing subsystem 125 may use a machine learning model to reconstruct an image, such as dual-encoder convolutional neural networks (CNNs) to process reflected and transmission image layers of an eye, Generative Adversarial Networks (GANs), transformer-based models to process dependencies that affect reflected images, or physics-informed neural networks (PINNs) to incorporate optical principals. In some embodiments, the testing subsystem 125 may generate an eye condition indication via the sandboxed vision testing application by creating a sequence of reconstructed images that approximate the image content based on the reflected image content data.

It should be understood that a reconstructed image may be a simplified version of an original image, and may include only a reconstruction of a brightness, a shape, or a position. For example, a reconstructed image may include a set of positions representing bright points or portions where an image moves with respect to prior frames. Alternatively, or additionally, the reconstructed image may include a combination of the shapes, positions, colors, or brightness of an originally presented content. For example, some embodiments may provide a sequence of images including a reflection of visible light from a person's eye to a machine learning model while the eye is presented with a video of a stationary man moving his hands. The machine learning model may then output a reconstruction of the video displaying the man moving his hands, a reconstruction of the hands alone, a set of values representing an overall brightness of the video or brightnesses of specific objects in the video, a set of points representing shapes detected in the video, timestamped points representing where the hands have moved over the course of the video, etc.

Some embodiments may generate a set of expected eye measurements based on the sequence of reconstructed images. These expected eye measurements represent the anticipated response of a healthy eye to the sequence of reconstructed images. For example, some embodiments may determine a set of expected eye responses (e.g., a twitch of the eye, a blink, a gaze change, a pupil dilation change, etc.) based on feedback data by directly providing the feedback data to the machine learning model. Alternatively, or additionally, some embodiments may generate a set of reconstructed images to a machine learning model, where the machine learning model may then predict a set of expected eye responses. For example, some embodiments may generate a sequence of vectors representing a set of reconstructed images of a video depicting a ball flying through the air. Some embodiments may then provide the sequence of vectors to a machine learning model as an input and obtain, as outputs of the machine learning model, a gaze change prediction that includes changes in eye's gaze positions as the eye tracks the ball and a speed of the change in gaze positions.

Some embodiments may generate an eye condition indication by using a machine learning model to compare the set of expected eye measurements with the eye-related feedback data. For example, some embodiments may determine that the eye-related feedback data indicates that an eye moved to track an object in motion after the object was within 40 degrees of the eye's visual field, but that the set of expected eye measurements indicates a predicted response once the object is within 60 degrees of the eye's visual field. Some embodiments may determine a difference between the predicted reaction and the actual reaction (e.g., as a difference in the time of gaze response, in the angle difference, in the magnitude of the eye response, etc.) provide this reaction difference to a machine learning model. This machine learning model may then output an eye condition indication that indicates a reduced visual field. Furthermore, it should be understood that operations of the testing subsystem 125 may be performed by one or more specific subsystems within the testing subsystem 125.

In some embodiments, the testing subsystem 125 may include the screening model subsystem 126, the diagnosing model subsystem 127, or other subsystems related to the hierarchical arrangement of different machine learning models. In some embodiments, the screening model subsystem 126 may use a machine learning model that receives feedback data as inputs and provides, as one or more outputs, a first prediction model output used to determine whether to trigger a more-complex model used by the diagnosing model subsystem 127. Various types of machine learning models or combinations of machine learning models may be used for outputting the first prediction model output, such as CNNs, RNNs, LSTMs, etc. For example, in some embodiments, the screening model subsystem 126 may directly provide a sequence of images of an eye obtained by inward-facing cameras to an RCNN to obtain labels for processing sequences of eye images, where the memory parameters of a machine learning models may be suited for tracking data across frames. By using a less-computationally-costly model such as the screening model subsystem 126 instead of a more-computationally-costly model such as the diagnosing model subsystem 127, computational resources may be conserved during regular operations of the client device 102.

In some embodiments the screening model subsystem 126 or the diagnosing model subsystem 127 may be sandboxed from data provided to or provided by the content presentation subsystem 123. In many cases, the sandboxed nature of a diagnosing model may require greater complexity. However, the conditions required for making use of these greater complexity models may not always be present (e.g., eyes are usually in a sufficiently “healthy” state). By using the screening model subsystem 126 to gatekeep the activation of greater complexity models, some embodiments may permit the use of one or more eye-condition-diagnosing models capable of operating in a sandboxed environment without requiring a constant, significant cost on device computing resources.

In some embodiments, a diagnosing model subsystem 127 may generate an eye condition indication based on the feedback data. In response to an output of the screening model subsystem 126 satisfying a set of triggering thresholds, some embodiments may provide the diagnosing model subsystem 127 with a set of inputs, or otherwise activate the diagnosing model subsystem 127. For example, the screening model subsystem 126 may determine a binary value based on an input feedback data stream. If the binary value indicates a potential issue (e.g., by being labeled “anomalous”), the screening model subsystem 126 may trigger the activation of the diagnosing model subsystem 127, which may be more complex or require additional resources.

In some embodiments, the model of the diagnosing model subsystem 127 and the screening model subsystem 126 may include models having the same architecture. For example, the screening model subsystem 126 may include a RCNN model, and the diagnosing model subsystem 127 may also include an RCNN model. The diagnosing model subsystem 127 may then output an eye condition indication, which may include an eye-related condition (e.g., “astigmatism,” “macular degeneration,” etc.), a general health condition (e.g., “diabetes,” “insufficient blood oxygen,” “jaundice”), etc.

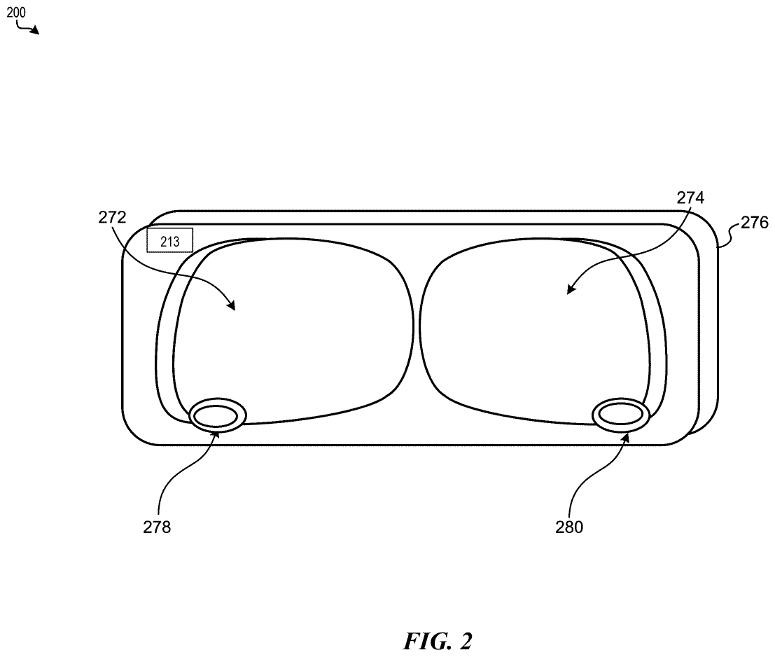

FIG. 2 is an example head-mounted device, in accordance with one or more embodiments. A head-mounted device 200 may be used as a content presentation device that may concurrently detect and analyze vision systems. This head-mounted device 200 may include two components: a left eyepiece 272 and a right eyepiece 274. Both eyepieces 272 and 274 are equipped with digital monitors that can either display or project reconstructed images directly to the wearer's eyes.

The digital monitoring system is housed within a frame structure 276, with the left eyepiece 272 and right eyepiece 274 carefully positioned to align with the wearer's orbital area. This arrangement enables the left eyepiece 272 and the right eyepiece 274 to both gather information and present visual data, with each component serving a distinct eye.

The system features a left inward-facing sensor 278 and a right inward-facing sensor 280. For example, the left inward-facing sensor 278 or the right inward-facing sensor 280 may utilize infrared technology for cornea tracking or other types of eye tracking activity. The left inward-facing sensor 278 and the right inward-facing sensor 280 may be placed in the lower sections of the left eyepiece 272 and the right eyepiece 274, respectively. The left inward-facing sensor 278 and the right inward-facing sensor 280 may be positioned in such a fashion as to avoid interfering with a user's field of vision. In some embodiments, the left inward-facing sensor 278 and the right inward-facing sensor 280 may be specifically oriented toward the expected pupil position to optimize tracking capabilities. Furthermore, in some embodiments, the left inward-facing sensor 278 and the right inward-facing sensor 280 may be embedded within the left eyepiece 272 and the right eyepiece 274, respectively to create a smooth interior surface.

In some embodiments, a first application executed by a set of processors 213 may present content via the left eyepiece 272 or the right eyepiece 274. As the content is projected to a user's eye, the left inward-facing sensor 278 or the right inward-facing sensor 280 may collect feedback data, such as feedback about a user's eyes responding to the presented content. The feedback data may then be provided by a second application also being executed by the set of processors 213, where the second application is sandboxed from the first application such that the presented content is not directly available to the second application. Such sandboxing may be common in various private or professional settings, where sandboxing may form an important aspect of device security and user privacy protection. In some embodiments, the second application may perform operations described in this disclosure to then determine one or more eye condition indications based on the feedback data. For example, the second application may provide feedback data collected by the left inward-facing sensor 278 and the right inward-facing sensor 280 to determine that a user is suffering from keratoconus.

In some embodiments, the set of processors 213 may execute one or more prediction models, where a prediction model may include machine learning models, statistical models, physics-based models, or rules-based models (e.g., a healthcare-provider-generated rules engine). In the context of machine learning models, various types of implementations of machine learning models may be used as machine learning models, unless otherwise stated, such as neural networks, random forests, and support vector machines. Less-resource-intensive machine learning model as content is being presented via the left eyepiece 272 or the right eyepiece 274. Some embodiments may collect feedback data about the user's eyes (e. g, feedback data collected by the left inward-facing sensor 278 or the right inward-facing sensor 280) as content is presented to a user. Some embodiments may then provide the feedback data to a less-resource-intensive machine learning model, which may trigger the use of a more-resource-intensive machine learning model, if certain criteria are satisfied, where the more-resource-intensive machine learning model may consume one or more than the less-resource-intensive machine learning model.

For example, some embodiments may provide the feedback data to a less-resource-intensive machine learning model that includes a two-layer neural network that consumes 5% of the processor resources provided by the set of processors 213. In some embodiments, the feedback data stream being provided to the less-resource-intensive machine learning model may then cause the less-resource-intensive machine learning model to output a value (e.g., a binary or categorical value indicating an eye status) that satisfies a triggering threshold. In response, the set of processors 213 may route the feedback data stream to the more-resource-intensive machine learning model, where some embodiments may provide historical data of the feedback to the more-resource-intensive machine learning model as well. In contrast to the less-resource-intensive machine learning model, the more-resource-intensive machine learning model may consume 20% of the processor resources provided by the set of processors 213. The more-resource-intensive machine learning model may then output an eye condition indication, such as “dry eye” or “keratoconus.”

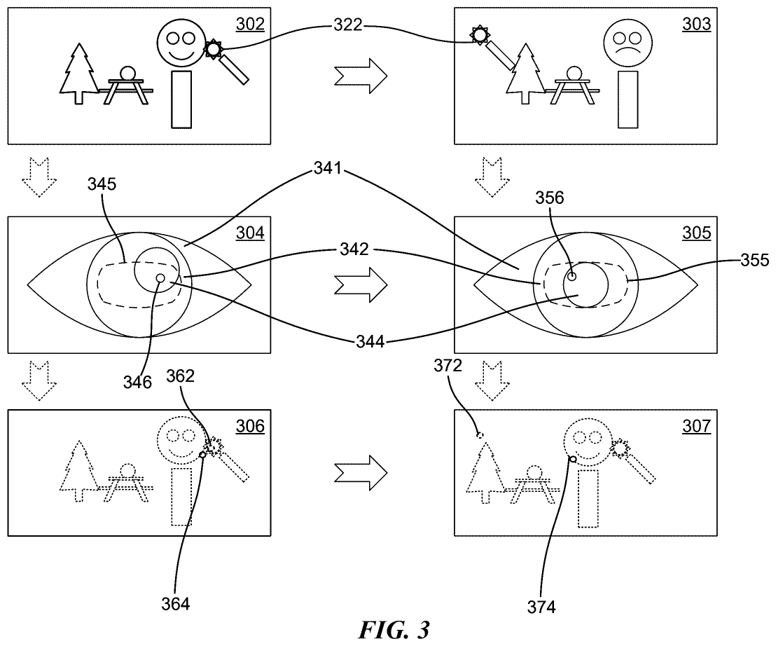

FIG. 3 shows an example conceptual diagram of content presentation being processed for detecting eye conditions, in accordance with one or more embodiments. In some embodiments, an application may present, to a user, image content that includes a first image 302 and then, at a later time, a second image 303. During the presentation of this image content, a bright object 322 may be moved from a first position in the first image 302 to a second position as shown in the second image 303. During this presentation, a user's eye may change its gaze to track the moving object.

Some embodiments may capture feedback data that captures eye responses during the presentation of image content, where an inward-facing sensor may capture a first eye image 304 while the first image 302 is being presented and capture a second eye image 305 while the second image 303 is being presented. The first eye image 304 shows the parts of a user's eye as the eye is being presented with the first image 302. The first image 302 depicts a sclera 341, an iris 342, and a pupil 344. The inward-facing sensor may further capture a reflection 345 that is a reflection of the first image 302, which includes a reflected point 346 that depicts the position of the bright object 322 presented in the first image 302. Similarly, the second eye image 305 shows the parts of the user's eye as the eye is being presented with the second image 303. The second eye image 305 also depicts the sclera 341, the iris 342, and the pupil 344, where the position of the pupil 344 may have moved in the second eye image 305 relative to the position of the pupil 344 in the first eye image 304. The inward-facing sensor may further capture a second image reflection 355 that is a reflection of the second image 303, which includes a second image reflected point 356 that depicts the position of the bright object 322 presented in the second image 303.

Some embodiments may provide the first eye image 304 to an image reconstruction subsystem to produce a first reconstructed image 306 that includes a first user interface location 362 representing a predicted focal point based on the bright object 322 in the first image 302. For example, some embodiments may provide the first reconstructed image 306 including the reflected point 346 to a first machine learning model in conjunction with other data (e.g., previous reconstructed images) to predict that an eye gaze location should fall upon the first user interface location 362. Similarly, some embodiments may provide the second eye image 305 including the point 356 to the first machine learning model or another portion of an image reconstruction subsystem to produce a second reconstructed image 307 that includes a second user interface location 372. The second user interface location 372 may represent a predicted focal point based on the position of the bright object 322 in the second image 303.

Some embodiments may detect a user's actual gaze positions using feedback data that includes the first eye image 304 or the second eye image 305. For example, some embodiments may determine that a set of response values indicate that the user's gaze actually fell upon the first actual gaze location 364. Some embodiments may determine that a set of response values indicate that the user's gaze actually fell upon a second actual gaze location 374. Some embodiments may then provide the locations of the first user interface location 362, the first actual gaze location 364, the second user interface location 372, and the second actual gaze location 374 to a second machine learning model. The second machine learning model may then output a predicted eye condition indication for an eye, where the eye condition indication may represent a diagnosis for one or more issues related to the eye.

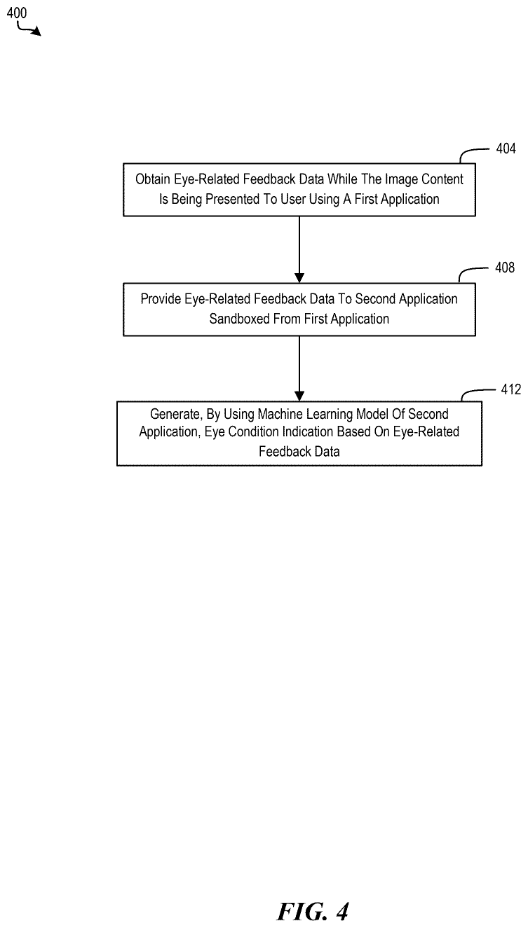

FIG. 4 shows a flowchart of a process 400 for detecting eye conditions during content presentation, in accordance with one or more embodiments. Some embodiments may obtain feedback data while presenting the image content to the user using a first application, as indicated by block 404. In some embodiments, a head-mounted device may concurrently execute a presenting application to present content and a sandboxed second application to collect eye-related feedback data to determine eye health. In connection with the presenting application displaying image content to a user via an inward-facing display of the head-mounted device, some embodiments may obtain eye-related feedback data through one or more inward-facing sensors. This feedback data includes (i) eye response data corresponding to the user's eye responses to the presented image content and (ii) reflected image content data corresponding to reflections off the user's eye while the image content is being displayed. For example, a head-mounted device may include an inward-facing eye-tracking camera that captures corneal reflections, pupillary movements, and oculomotor behavior as the user navigates through a gaming environment. In some embodiments, the camera may include infrared sensors that may detect specular reflections from the corneal surface or the dynamic changes in iris patterns during gameplay.

Some embodiments may collect infrared measurements. For example, some embodiments may obtain eye-related feedback data by collecting a set of infrared reflection measurements of the infrared light reflected from the eye. As described elsewhere in this disclosure, some embodiments may then use these infrared reflection measurements to generate the eye condition indication. For example, inward-facing cameras may use a set of infrared diodes or other infrared-emitting devices to emit infrared light. The infrared light may illuminate a user's eyes with infrared light while specialized sensors capture the reflected patterns. In some embodiments, the invisible infrared light may create glints on a cornea or another portion of the eye and highlight pupil position. Some embodiments may then process these infrared reflections as part of the feedback collected by an inward-facing sensor. In some embodiments, the infrared light being projected may be projected by a special infrared light projecting device (e.g., an infrared light-emitting diode) that is separate from a visual display of a head-mounted device. Alternatively, some embodiments may project infrared light with an inward-facing display, such as the same inward-facing display used to present content to a user. For example, some embodiments may use a display that includes embedded infrared light emitters that may then project infrared light onto a user while also presenting image content in the visible light spectrum.

It should be understood that some embodiments may collect more limited data using the inward-facing sensors or expanded data. For example, some embodiments may collect pupil responses without collecting reflection data, collect eye reflection data without collecting pupil responses, etc. Furthermore, while some embodiments may use a light-emitting device or another electronic component to project infrared light that is then reflected off an eye and collected by a sensor, some embodiments may collect eye-related feedback data without using a light-emitting device by collecting data provided by an inward-facing display or a natural light sent through a lens.

Some embodiments may provide eye-related feedback data to a second application sandboxed from the first application, as indicated by block 408. Some embodiments may execute a testing application concurrently with content-presenting applications, where the testing application is sandboxed from the content presenting application such that data from the content-presenting application is not directly available to the sandboxed application.

Some embodiments may obtain, with the second application, a set of display utilization data of the head-mounted device while the image content is being projected. For example, some embodiments may provide data to a sandboxed application via a graphics application program interface (API) that renders approved content or anonymized content generated by or sent to third-party applications. Some embodiments may then determine a set of brightness values or other graphics-based values based on the set of display utilization data. Some embodiments may then determine the presence of an eye-related anomaly by comparing the set of brightness values with the measured brightness, where the measured brightness may be determined from eye-related feedback data. For example, some embodiments may determine that eye-related feedback data indicates a brightness value of the screen that is equal to 0.3 with respect to a maximum brightness value, and further that graphics data provided via the API indicates that the brightness value of the screen is equal to 0.5. Some embodiments may use this discrepancy as a representation that an anomalous eye condition may be applicable to a user's eye.

Some embodiments may obtain a set of display utilization data of the head-mounted device while image content is being projected. Some embodiments may then determine a set of brightness values or other graphics-based values based on the set of display utilization data and then generate a set of expected response values based on the set of brightness values or other graphics-based values. For example, some embodiments may generate an expected response of rapid pupil constriction in response to a sudden increase in brightness. Some embodiments may classify an eye condition based on a difference between this expected response for an eye and a measured response of the eye. For example, if a dilation is too slow, does not occur, or does not dilate to an expected size, some embodiments may provide feedback data and the expected response data to a machine learning model that indicates one or more eye conditions.

Some embodiments may detect a measured head shift time via the orientation sensors of the head-mounted device, where the head shift may indicate a shift from a first head orientation to a second head orientation while the image content is being presented. Some embodiments may use an inertial measurement unit that combines data from accelerometers and gyroscopes to track rotational movement across three axes and then determine stable orientation estimates to determine head orientation. As described elsewhere in this disclosure, some embodiments may then use this information as an input to determine an eye condition indication.

Some embodiments may generate an eye condition indication based on eye-related feedback data using a machine learning model of the second application, as indicated by block 412. Some embodiments may use a machine learning model that is directly trained to predict eye condition indications based on the feedback data. For example, the machine learning model may include a convolutional neural network. Some embodiments may obtain input images from one or more inward-facing cameras and provide them as inputs to a trained neural network that then directly outputs eye condition indications, where the eye condition indications may indicate conditions such as dry eye or a specific type of dry eye condition, keratoconus, marginal degeneration, ectasia, keratoglobus, or another eye-related condition.

Some embodiments may provide the feedback data to a testing application that is sandboxed from a content-presenting application. For example, some embodiments may provide feedback data collected by a set of eye-tracking sensors through a secure channel to a sandboxed application. In some embodiments, the feedback data may include pupil position data, reflection data, or other types of data. The system's security layer validates the data stream before allowing it to cross the sandbox boundary. The gaming application receives only pre-approved sensor data types through designated APIs to maintain isolation.

Some embodiments may generate simulated input data, such as a first set of reconstructed images, based on the eye-related feedback data. In generating the eye condition indication, the system may obtain a set of display utilization data of the head-mounted device with the second application while the image content is being projected. This display utilization data is then transformed into a second set of reconstructed images. The system determines a set of differences between the first set of reconstructed images and the second set of reconstructed images, and the eye condition indication is determined based on these differences.

Some embodiments may determine a set of user interface locations for visual stimuli based on simulated input data to determine stimuli positions that would cause gaze changes. For example, some embodiments may reconstruct images based on reflections of light from an eye. Some embodiments may then determine likely focal points based on the reconstructed images. Additionally, some embodiments may determine the set of user interface locations based on a sequence of frames of the simulated input data while the image content is being presented by the head-mounted device. For example, some embodiments may track the motion of objects, brightness, changes in brightness, and other elements of a set of images to predict one or more locations that an eye would be expected to focus on.

Some embodiments may generate a set of expected response values based on user interface locations and predict changes in gaze location using eye motion. Additionally, some embodiments may use a machine learning model to generate expected eye response values, such as the speed and trajectory of gaze changes, and determine eye conditions based on these eye response values. By comparing the expected eye response values with actual measured responses from feedback data, some embodiments may identify visual field impairments if the eye does not respond appropriately to visual stimuli.

As described in this disclosure, an impairment in the visual field includes a failure to detect changes outside a visual field boundary. For example, in some embodiments, a visual field boundary may be set to a limit of human perception. The boundary may encompass a visual field that is less than 110 degrees in a temporal direction, less than 60 degrees in a nasal direction, less than 60 degrees in a superior direction, or less than 75 degrees in an inferior direction. It should be understood that some embodiments may use other boundaries for a visual field, such as a visual field that is less than 100 degrees in the temporal direction, less than 50 degrees in a superior direction, or less than 70 degrees in an inferior direction. Furthermore, some embodiments may use a user-specific stored boundary for a visual field to detect whether a user responds to stimuli that is presented in what should be the user's visual field.

As described elsewhere, some embodiments may use head shift information to determine an eye-related indication. Some embodiments may generate a predicted head shift time based on the feedback data. For example, some embodiments may use operations described in this disclosure to predict when a sudden change in the visual display of content (e.g., change in brightness, change in shape, sudden appearance or disappearance of one or more objects) is predicted to cause a response in head shifts. Some embodiments may then generate an eye condition indication using both the predicted head shift time and the measured head shift time by comparing the two values. For example, if a shift is expected to occur but does not, some embodiments may determine that an eye condition for an eye is a likely source of the discrepancy and generate a corresponding eye condition indication for that eye.

Some embodiments may use one or more rules-based systems to determine an eye condition. For example, some embodiments may use a machine learning model to either reconstruct presented content based on feedback data or predict an expected eye response to the presented content. Some embodiments may then compare the expected eye response to an actual eye response as determined based on the feedback data.

Furthermore, some embodiments may use multiple machine learning models when generating an eye condition indication, such as by performing one or more operations similar to or the same as those described elsewhere in this disclosure. For example, some embodiments may determine a first prediction model output using one or more operations described for block 708. Some embodiments may then generate an eye condition indication using a second prediction model using one or more operations described for block 720 based on one or more criteria being satisfied by the first prediction model output.

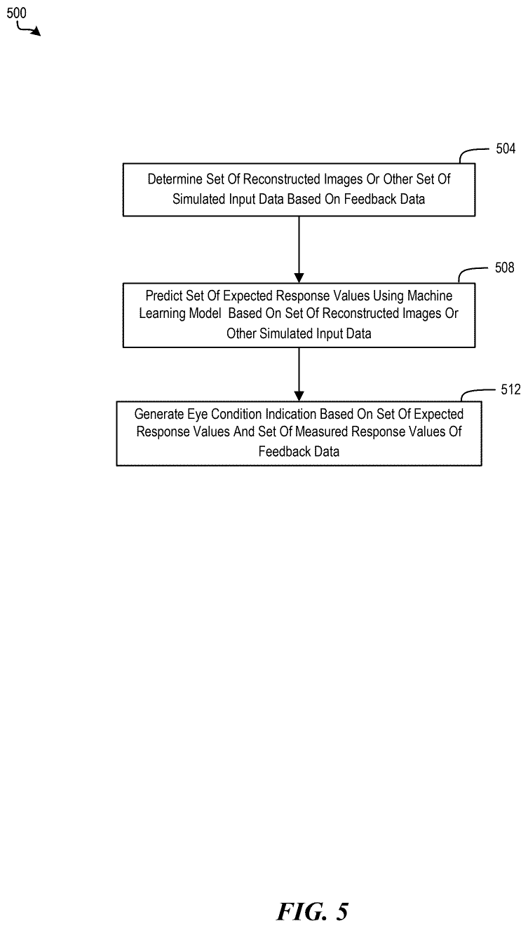

FIG. 5 shows a flowchart of a process 500 for detecting eye conditions by generating simulated input data based on feedback data, in accordance with one or more embodiments. Some embodiments may determine a set of reconstructed images or other set of simulated input data based on feedback data, as indicated by block 504. Some embodiments may collect the feedback data using one or more operations described in this disclosure, such as operations described for block 404. For example, some embodiments may collect feedback data from inward-directed sensors. Some embodiments may then use this feedback data to generate simulated input data representing data being displayed to a user, such as a set of reconstructed images representing images being presented to a user. For example, a sandboxed application may generate simulated input data by reconstructing image content being presented to a user.

Some embodiments may generate a first set of reconstructed images based on the eye-related feedback data, such as reconstructing image data from the reflection on an eye. For example, an inward-facing camera may capture high-resolution images of corneal reflections and apply a set of inverse geometric transformations to unwarp the distorted image. Some embodiments may further use one or more machine learning models or other types of subsystems to correct for an eye's curvature and other possible sources of error to generate a reconstructed set of images. Furthermore, some embodiments may obtain a set of display utilization data of the head-mounted device with the second application while the image content is being projected and use this utilization data to generate a second set of reconstructed images. Some embodiments may then determine a set of differences between the first set of reconstructed images and the second set of reconstructed images and then determine an eye condition indication based on these differences. For example, some embodiments may determine that, based on a type of warping detected between the first set of reconstructed images and the second set of reconstructed images, the cornea shows a physical distortion in the form of keratoconus.

Some embodiments may use a machine learning model to predict a set of expected response values based on the set of reconstructed images or other simulated input data, as indicated by block 508. Some embodiments may predict a set of expected response values based on reconstructed images or other simulated input data that indicates user interface locations that could cause responses in a user's eyes. For example, some embodiments may first determine a set of predicted focal points based on a set of reconstructed images. Some embodiments may then predict, as an expected response value, a change in a gaze location based on eye motion tracking the predicted focal point. Additionally, some embodiments may use a machine learning model to generate a predicted eye response value for a gaze change to a new location within the set of user interface locations, where the set of user interface locations includes the predicted eye response value. For example, some embodiments may use a machine learning model that outputs a predicted eye response value such as a speed of gaze change, the trajectory of a gaze change, etc. Furthermore, it should be understood that while the above example describes using a machine learning model to predict a set of expected response values, some embodiments may use other types of prediction models, such as a statistical model or a rules-based model.

Some embodiments may generate an eye condition indication based on the set of expected response values and a set of measured response values of the feedback data, as indicated by block 512. Some embodiments may then determine an eye condition indication using a set of expected response values related to the behavior of an eye. Some embodiments determine how a difference between this expected response value with an actual measured response is determined from feedback data and predict an eye condition based on the actual measured response. When determining the reaction difference, some embodiments may determine a reaction difference between the predicted eye response value and an actual eye response value indicated by the eye-related feedback data. For example, some embodiments may determine that an eye that would have been expected to move to a new location based on machine learning model outputs may instead be affected by a visual field impairment based on a reaction difference indicating that the eye did not respond appropriately to visual stimuli. Some embodiments may use a machine learning model to predict an eye condition indication by providing, as inputs, the set of expected response values and the set of measured response values to the machine learning model, where the machine learning model may output a set of values indicating one or more eye conditions. Alternatively, some embodiments may use a rules-based model and provide the rules-based model with the set of expected response values and the set of measured response values to determine one or more eye conditions.

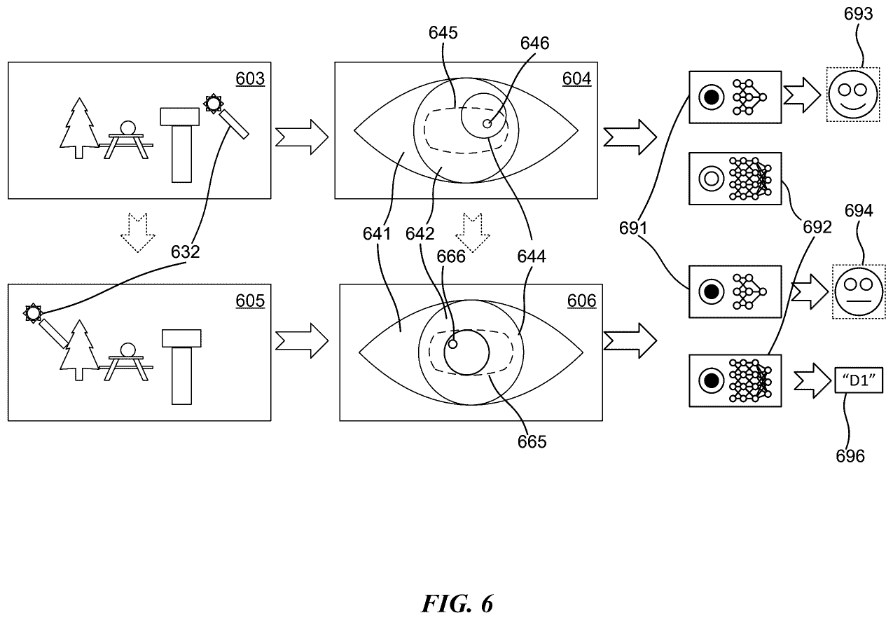

FIG. 6 shows an example conceptual architecture for preserving computer resources while detecting eye conditions, in accordance with one or more embodiments. In some embodiments, an application may present, to a user, image content that includes a first image 603 and then, at a later time, a second image 605. Some embodiments may capture feedback data that captures eye responses during the presentation of image content, where an inward-facing sensor may capture a first eye image 604, while the first image 603 is being presented and capture a second eye image 606, while the second image 605 is being presented. The first eye image 604 shows the parts of a user's eye as the eye is being presented with the first image 603. The first eye image 604 includes a sclera 641, an iris 642, and a pupil 644. The inward-facing sensor may further capture a reflection 645, which includes a reflected point 646 that depicts the position of a bright object 632 presented in the first image 603. The inward-facing sensor may further capture a reflection 645 that would be a reflection of the first image 603, where the reflection 645 includes a reflected point 646 that depicts the position of the bright object 632 presented in the first image 603. Similarly, the second eye image 606 shows the parts of the user's eye as the eye is being presented with the second image 605. The second eye image 606 also depicts the sclera 641, the iris 642, and the pupil 644, where the pupil 644 has been repositioned to a second position in response to changes depicted in the transition from the first image 603 to the second image 605. Additionally, the inward-facing sensor may further capture a reflection 665 that would be a reflection of the second image 605, where the reflection 665 includes a reflected point 666 that depicts the position of the bright object 632 presented in the second image 605.

As the position of the bright object 632 changes from the position shown in first image 603 to the position shown in second image 605, a user's eye may change its gaze to follow the bright object 632. During the presentation of the first image 603, the first eye image 604 or data derived from the first eye image 604, may be provided as inputs to a first machine learning model 691, where the first machine learning model 691 may be less complex or less resource-intensive than the second machine learning model 692. In response to determining that output 693 of the first machine learning model 691 does not satisfy a triggering threshold, some embodiments may refrain from using a second machine learning model 692.

During the presentation of the second image 605, the second eye image 606 or data derived from the second eye image 606, may be provided as inputs to the first machine learning model 691. In some embodiments, based on the data provided by the second eye image 606, historical data in a data stream of the eye shown in the first eye image 604, or other data, the first machine learning model 691 may output a second prediction model output 694. Some embodiments may determine that the second prediction model output 694 of the first machine learning model 691 satisfies a triggering threshold. In response, some embodiments may activate the second machine learning model 692 by providing, as inputs, the second eye image 606 to the second machine learning model 692 to produce an eye condition indication 696. Furthermore, while not shown in FIG. 6, some embodiments may stop monitoring feedback data or data derived from the feedback data with the first machine learning model 691 once the second machine learning model 692 is used to monitor feedback data or data derived from the feedback data.

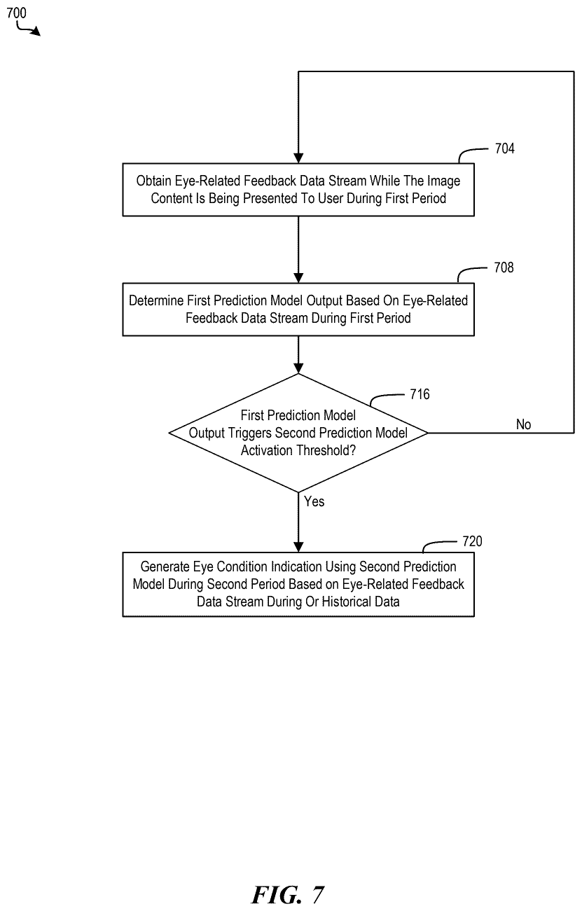

FIG. 7 shows a flowchart of a process 700 for detecting eye conditions during content presentation, in accordance with one or more embodiments. Some embodiments may obtain eye-related feedback data while image content is being presented to the user during a first period, as indicated by block 704. While presenting image content through an inward-facing display of a head-mounted device, some embodiments obtain a feedback data stream from a set of inward-facing sensors during the presentation. For instance, while a user is watching a video on a VR headset, the headset system may use one or more inward-facing sensors to gather data on the user's eye responses and reflections. Furthermore, the feedback data stream may include various types of data from one or more sensors, such as eye response data or reflection data collected during the presentation of the image content in a first period. For example, while a user watches a video on a VR headset, the system may use inward-facing camera sensors to collect a feedback data stream that includes eye response data and reflection data during the video playback.

Some embodiments may project infrared light onto an eye, using an infrared emitting device of the head-mounted device and then retrieve the reflected infrared light data, where feedback data stream includes the infrared light reflection data. For example, while a user is playing a video game on a VR headset, a system may project infrared light onto a user's cornea using an infrared emitting device of the head-mounted device. The system may then retrieve the reflected infrared light data, which includes information about the cornea's response. The use of this infrared light in eye-tracking systems may allow for accurate and non-intrusive monitoring of eye movements in low-light conditions such as the space between the face and a head-mounted device.

When collecting data, some embodiments may disable a sensor and reactivate the sensor based on data collection conditions. Some embodiments may detect that the visible light output of the inward-facing display is too low (e.g., and thus would not be able to obtain meaningful metrics of eye behavior) and, in response, deactivate the infrared emitting device used to project the infrared light. For example, some embodiments may detect that a visible light output of the inward-facing display is below a light threshold of 5 lux and, in response, deactivate an infrared emitting diode device used to project the infrared light. It should be understood that other values of the light threshold may be used, such as a value less than or equal to 10 lux, a value less than or equal to 20 lux, a value less than or equal to 100 lux, etc. Some embodiments may then reactivate a previously deactivated infrared emitting device upon a determination that the light threshold is exceeded. Alternatively, some embodiments may stop a use of one or more prediction models (e.g., a screening prediction model or a second prediction model that is activated based on a result of the screening prediction model) based on a determination a light-related value has fallen below the light threshold. For example, some embodiments may stop the use of a screening prediction model by stopping more data from a data stream from being provided as inputs for the screening prediction model after determining that the light output of a display is below a light threshold.

Some embodiments may predict a reflection brightness for light being reflected off an eye (e.g., off a cornea) based on feedback data that includes measured brightness value. Some embodiments may then compare this reflection brightness with a brightness threshold that acts as a minimum brightness threshold. In response to determining that the minimum brightness threshold is violated, some embodiments may begin providing one or more data streams to a greater-complexity prediction model described elsewhere in this disclosure.

Some embodiments may retrieve a content data stream used to present image content via a network connection of the head-mounted device and present the image content obtained from the content data stream. As described elsewhere in this disclosure, in some embodiments, one or more prediction models may be part of a testing application that is sandboxed from content-presenting applications, such that a content data stream is not directly available to the testing application. Alternatively, in some embodiments, a content-presenting application is not sandboxed from a testing application, such that the content data stream itself may be accessible to the testing application. Thus, in such embodiments, an input for one or more prediction models described in this disclosure may include the content data stream or data derived from the content data stream (e.g., images from the content data stream). For instance, while a user is watching a movie on a VR headset, some embodiments may stream video content from an online service and display it on the inward-facing display of the headset. Some embodiments may provide this video content as an input for a testing application.

Some embodiments may determine a first prediction model output based on the eye-related feedback data collected during the first period, as indicated by block 708. Using the feedback data stream, some embodiments may determine the output of a first prediction model. For example, a head-mounted system may use inward-facing sensors to collect a feedback data stream that includes eye tracking data and reflection data during the video playback. Some embodiments may then determine an output by inputting this feedback data stream into a screening machine learning model of a second application that is sandboxed from the first application. An output of the screening machine learning model may include a first condition indication, a general indication of health, a numerical value indicating confidence in eye health, or the presence of an eye issue, etc.

Some embodiments may determine a binary value by using the feedback data stream as input for a screening machine learning model in a second application that is isolated from the first application. For example, a head-mounted device may use a video application to present image content and then analyze eye tracking data with a separate health monitoring application running concurrently but independently from the video application. Based on the analysis, some embodiments may generate a binary value indicating whether the user's eye condition is ‘healthy’ or ‘unhealthy.’

Some embodiments may, while presenting image content, generate predicted reflection image data using the first prediction model by providing image data of the image content to the first prediction model. Some embodiments may then determine a set of differences between the predicted reflection image data and the actual feedback data stream. Based on these differences, some embodiments may then determine a first prediction model output used to determine whether to activate a greater-complexity prediction model. Various methods may be used to determine image differences, such as a Squared Error method that calculates pixel-by-pixel differences to provide a basic numerical comparison, a Structural Similarity Index method that evaluates differences in luminance, contrast, and structural information, or feature extraction techniques. Some embodiments may use CNN or perceptual loss functions to determine differences. For example, some embodiments may compare a predicted image and an actual image using both a Squared Error method and a CNN to determine a set of numerical values as a set of difference values. This set of difference values may be used as a prediction model output, be included as part of the prediction model output, or be used to compute a prediction model output (e.g., by summing values, determining a weighted sum, etc.). As described elsewhere in this disclosure, some embodiments may then determine that a set of numerical values that exceeds a maximum threshold, or is less than minimum threshold triggers a set of criteria that, if triggered, causes the use of a second prediction model that is more complex than the first prediction model. Such operations may help provide an accurate diagnosis of eye health due to the relationship between a predicted reflection for a healthy cornea or any other part of an eye, where deviations from this predicted reflection may indicate the presence of aberrations, abnormal eye wetness conditions, or another health-related issue.

Some embodiments may, during the presentation of image content, produce predicted pupil reaction data or predicted eye movement data by supplying the image data of the content or data of the content to the first prediction model. These embodiments may then identify a series of discrepancies between the predicted pupil reaction data or predicted eye movement data and the actual feedback data stream. Based on these discrepancies, the embodiments may then determine a first prediction model output to decide whether to engage a more complex prediction model. For example, some embodiments may predict a pupil contraction, a pupil expansion, a shift in gaze, or another eye-related reaction based on image content that the user is predicted to see. Some embodiments may then compare this prediction with the measured eye response collected in feedback data to determine a prediction model output based on any detected discrepancies. For example, a prediction model may output “unhealthy” based on a discrepancy indicating that a predicted pupil contraction responding to an increase in image brightness was not observed in measured eye-related data. Similarly, a prediction may output “unhealthy” based on a discrepancy indicating that a predicted eye movement responding to a moving object was not observed in measured eye-related data.

Some embodiments may determine the reflection surface area from the feedback data stream. For example, some embodiments may use a direct mapping method that applies geometric transformations to map the reflection back to a set of original screen dimensions based on known corneal curvature and distance parameters. Alternatively, some embodiments may use a feature-based method that identifies key points in the corneal reflection and uses these reference points to reconstruct an original screen dimension, and position with perspective transformation algorithms. Some embodiments may then determine whether the computed surface area meets or exceeds a size threshold, where the size threshold may be obtained as a default value or calculated from a prior calibration operation. Based on the result of this determination based on the computed surface area and the size threshold, some embodiments may provide the feedback data stream to a screening prediction model to perform further predictions. Alternatively, or additionally, some embodiments may provide the feedback data stream to a greater-complexity machine learning model, or another type of prediction model based on the result of this determination, where such a criteria may augment or circumvent other operations described as triggering the use of a greater-complexity prediction model.

Some embodiments may determine whether the first prediction model output triggers a set of activation criteria for the second prediction model, as indicated by block 716. In some embodiments, the first prediction model output may be or may include a binary output in that only one of two outputs are possible. For example, some embodiments may determine a binary value, by inputting a feedback data stream, including eye tracking data and reflection data into a screening machine learning model to generate a binary value, indicating whether the user's eye condition is ‘healthy’ or ‘unhealthy’. In response to a determination that the screening machine learning model has output “unhealthy,” some embodiments may determine that the set of activation criteria for the second prediction model has been triggered.

In some embodiments, the first prediction model output may be or may include a categorical output that may be one of more than two possible outputs, where triggering a set of activation criteria may include the first prediction model output being one of a set of triggering categories. For example, some embodiments may provide, as learning model inputs, feedback data to a first machine learning model that outputs a categorical output, indicating the user's eye condition as ‘normal’, ‘mild’, ‘moderate’, or ‘severe’. The system then determines whether this categorical output triggers a set of activation criteria for the second prediction model, such being one of a set of triggering categories. For example, some embodiments may treat the categories ‘mild’, ‘moderate’, or ‘severe’ as triggering categories, such that, if the first prediction model output is any category value other ‘normal,’ some embodiments may determine that the set of activation criteria for the second prediction model has been triggered.

In some embodiments, the first prediction model output may be or may include numerical output. For example, some embodiments may provide eye tracking data or other data in a feedback data stream to a first machine learning model that then generates a numerical value, indicating the user's eye condition on a scale from 0 to 100, where 0 represents poor eye health and 100 represents excellent eye health. The system then determines whether this numerical output triggers a set of activation criteria for the second prediction model, such being less than a triggering threshold. For example, if the threshold is equal to 90 and the model output value is equal to 80, some embodiments may determine that the set of activation criteria for the second prediction model has been triggered.

In response to a determination that the first prediction model output triggers the set of activation criteria, operations of the process 400 may proceed to operations described for block 720. Otherwise, operations of the process 400 may return to operations described for block 704, as additional feedback data is collected in a data stream.

Some embodiments may use a second prediction model to generate an eye condition indication during a second period, based on the feedback data stream or historical data, including feedback data gathered during the first period, as indicated by block 720. Some embodiments may collect historical data related to feedback gathered during the first period, all while the first application continues to present the image content. For example, some embodiments may have used inward-facing sensors to collect eye response data and reflection data via a feedback data stream during a first hour, while a user watches a video on a head-mounted device. After determining that a set of criteria for activating a greater-complexity machine learning model is triggered, some embodiments may provide, as a set of inputs, the feedback data stream to a greater-complexity machine learning model during a second period that begins when the set of criteria is triggered. Furthermore, the greater-complexity machine learning model may also process historical data, such that some embodiments may provide, as a set of inputs, both current data via the feedback data stream, and historical data including the eye response data and reflection data gathered during the first period. By using both historical and current data, the greater-complexity machine learning model may be provided with greater context and output a more accurate eye condition diagnosis for an eye. Furthermore, by using this hierarchical arrangement between a screening machine learning model and a greater-complexity machine learning model, system resources may be conserved by activating the greater-complexity machine learning model, only when a set of criteria is triggered.

When determining a set of eye condition indications, some embodiments may determine an indication that indicates conditions such as keratoconus, marginal degeneration, ectasia, or keratoglobus. Operations described in this disclosure may be especially beneficial for detecting such conditions due to the manifestations of such conditions. For example, some embodiments may use a first greater-complexity machine learning model to detect the presence of a bulge pattern indicative of keratoconus or keratoglobus, where a bulge pattern may be visually indicated by a localized warping of a portion of a reflected image. Some embodiments may use a second greater-complexity machine learning model to detect reflection changes in corneal reflections indicative of marginal degradation.

Some embodiments may, as part of the process for providing inputs to the second prediction model, select a particular prediction model instead of multiple other prediction models stored in a set of memory. For example, some embodiments may use a screening prediction model to obtain the categorical output “non-responsive to stimuli.” In response, some embodiment may select, as a greater-complexity prediction model, a prediction model trained to output eye condition indications related to a failure to respond to stimuli. If instead some embodiments use a screening prediction model to obtain the categorical output “abnormal reflection,” some embodiments may select an alternative prediction model trained to output eye condition indications related to an aberration in an eye reflection. Furthermore, in some embodiments, the output of a screening prediction model may be mapped to one or more eye conditions of one or more sets of eye conditions. Some embodiments may then use this mapping to select a first greater-complexity prediction model in lieu of other greater-complexity prediction models, based on associations between the first greater-complexity prediction model and one or more eye conditions. For example, some embodiments may use a first machine learning model or other screening prediction model to generate an output “bulge,” which may be mapped to a first set of eye conditions such as “keratoconus” or “keratoglobus,” where both conditions map to a first greater-complexity model. The model may have also been able to output “thinning” that is mapped to a second set of eye conditions that includes “marginal degeneration” and “anomalous thinning,” where the second set of eye conditions is associated with an alternative greater-complexity model. Some embodiments may then select the first greater-complexity prediction model in lieu of the alternative greater-complexity model, based on the first machine learning model outputting the value “bulge,” the association between “bulge” and the first set of eye conditions, and the association between the first set of eye conditions and the first greater-complexity model.

Some embodiments may help preserve additional computing resources by stopping an execution of the first prediction mode. For example, some embodiments may stop the execution of the first prediction mode after triggering the activation of a greater-complexity prediction model. For example, some embodiments may determine numeric score representing eye health by inputting a feedback data stream, a screening machine learning model, and determine that the numeric score satisfies an activation score threshold or another set of criteria (e.g., the numeric ranges from 1-10, and a score greater than 2 satisfies the activation score threshold). If the activation score threshold is activated, some embodiments may activate a greater-complexity prediction model to perform further analysis. Once the greater-complexity prediction model is activated, some embodiments may stop the execution of the first prediction model to conserve computing resources, such as processor resources, volatile memory capacity, or non-volatile memory capacity.

Some embodiments may help preserve additional computing resources by stopping an execution of the second prediction model. For example, after activating the greater-complexity prediction model by providing input data to the greater-complexity prediction model, some embodiments may determine that the greater-complexity prediction model generates a first eye condition indication that indicates a healthy eye. Some embodiments may then determine that a healthy eye condition has been indicated for at least a duration threshold (e.g., a value less than or equal to 10 seconds, a value less than or equal to one minute, a value less than or equal to one hour, a value less than or equal to one day, etc.). In response to determining that the outputs of the greater-complexity model have been indicating a healthy eye condition for at least a duration threshold, some embodiments may stop using the greater-complexity prediction model.

Some embodiments may obtain information indicating that one or more stimuli known to trigger a strong response in a healthy eye is to be presented to a user and, in response to receiving this information, automatically start the machine learning model. For example, some embodiments may, via a network connection (e.g., a wired or wireless network connection), receive content data that includes stimuli and an indication of when the stimuli are being presented. Some embodiments may receive a first indication of a stimulus in stored image content via a network connection, where presenting the image content involves displaying this stored image content. For example, some embodiments may receive an indication of a stimulus in the stored video content via a network connection, while a user is watching a movie on a VR headset. The system then presents the stored video content, including the indicated stimulus, on the inward-facing display of the headset. Some embodiments may receive a message, a second indication, or have it known from the first indication that the stimulus should be presented within a specific time threshold. Some embodiments may then automatically provide feedback data stream or historical data to a greater-complexity prediction model directly, such that the activation of the greater-complexity prediction model occurs independently of a prediction model output generated by a screening prediction model. For example, a head-mounted device may receive a message indicating that a specific scene with a bright flash will occur within the next 10 seconds. Based on this message, some embodiments may automatically begin to provide a feedback data stream to a greater-complex prediction model for analysis, independently of the initial prediction model output.

The above-described embodiments of the present disclosure are presented for purposes of illustration and not of limitation, and the present disclosure is limited only by the claims which follow. Furthermore, it should be noted that the features and limitations described in any embodiment may be applied to one or more other embodiments herein, and flowcharts or examples relating to one embodiment may be combined with any other embodiment in a suitable manner, done in different orders, or done in parallel. In addition, the systems and methods described herein may be performed in real time. It should also be noted that the systems and/or methods described above may be applied to, or used in accordance with, other systems and/or methods. Furthermore, not all operations of a flowchart need to be performed. In addition, the systems and methods described herein may be performed in real time. It should also be noted that the systems and/or methods described above may be applied to, or used in accordance with, other systems and/or methods.

Furthermore, the computing devices described in this disclosure may be any type of computing device unless otherwise stated, including, but not limited to, a laptop computer, a tablet computer, a hand-held computer, and/or other computing equipment (e.g., a server), including “smart,” wireless, wearable, and/or mobile devices. For example, while the client device 102 of FIG. 1 may be a head-mounted device, another type of mobile computing device may be possible. Furthermore, the embodiments described in this disclosure may include an individual device that performs some or all the operations described in this disclosure. Alternatively, other embodiments may include multiple computing devices acting collectively to perform some or all the operations described in this disclosure.

As used in the specification and in the claims, the singular forms of “a,” “an,” and “the” include plural referents unless the context clearly dictates otherwise. In addition, as used in the specification and the claims, the term “or” means “and/or” unless the context clearly dictates otherwise. Additionally, as used in the specification, “a portion” refers to a part of, or the entirety (i.e., the entire portion), of a given item (e.g., data) unless the context clearly dictates otherwise. Furthermore, a “set” may refer to a singular form or a plural form, such that a “set of items” may refer to one item or a plurality of items.

In some embodiments, the operations described in this disclosure may be implemented in a set of processing devices (e.g., a digital processor, an analog processor, a digital circuit designed to process information, an analog circuit designed to process information, a state machine, and/or other mechanisms for electronically processing information). The processing devices may include one or more devices executing some or all of the operations of the methods in response to instructions stored electronically on one or more non-transitory, machine-readable media (e.g., a set of machine-readable storage media), such as an electronic storage medium. Furthermore, the use of the term “media” may include a single medium or combination of multiple media, such as a first medium and a second medium. One or more non-transitory machine-readable media storing instructions may include instructions included on a single medium or instructions distributed across multiple media. For example, non-transitory media may include program instructions that are written as source files or written in machine-executable program code. The processing devices may include one or more devices configured through hardware, firmware, and/or software to be specifically designed for the execution of one or more of the operations of the methods.

In some embodiments, the various computer systems and subsystems illustrated in FIG. 1 or FIG. 2 may include one or more computing devices that are programmed to perform the functions described herein. The computing devices may include one or more electronic storages (e.g., a set of databases accessible to one or more applications depicted in the system 100), one or more physical processors programmed with one or more computer program instructions, and/or other components. For example, the set of databases may include one or more relational databases. Alternatively, or additionally, the set of databases or other electronic storage used in this disclosure may include one or more non-relational databases.

The computing devices may include communication lines or ports to enable the exchange of information with a set of networks (e.g., a network used by the system 100) or other computing platforms via wired or wireless techniques. The network may include the internet, a mobile phone network, a mobile voice or data network (e.g., a 5G or Long-Term Evolution (LTE) network), a cable network, a public switched telephone network, or other types of communication networks or combination of communication networks. A network described by devices or systems described in this disclosure may include one or more communications paths, such as Ethernet, a satellite path, a fiber-optic path, a cable path, a path that supports internet communications (e.g., IPTV), free-space connections (e.g., for broadcast or other wireless signals), Wi-Fi, Bluetooth, near field communication, or any other suitable wired or wireless communications path or combination of such paths. The computing devices may include additional communication paths linking a plurality of hardware, software, and/or firmware components operating together. For example, the computing devices may be implemented by a cloud of computing platforms operating together as the computing devices.

Each of these devices described in this disclosure may also include electronic storages. The electronic storage may include one or more non-transitory machine-readable media (e.g., storage media) that electronically stored information. The storage media of the electronic storages may include one or both of (i) system storage that is provided integrally (e.g., substantially non-removable) with servers or client computing devices, or (ii) removable storage that is removably connectable to the servers or client computing devices via port (e.g., a USB port, a firewire port, etc.) or drive (e.g., a disk drive, etc.). The electronic storages may include one or more optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. The electronic storages may include one or more virtual storage resources (e.g., cloud storage, a virtual private network, and/or other virtual storage resources). An electronic storage may store software algorithms, information determined by the processors, information obtained from servers, information obtained from client computing devices, or other information that enables the functionality as described herein.

The processors may be programmed to provide information processing capabilities in the computing devices. As such, the processors may include one or more of a digital processor, an analog processor, a digital circuit designed to process information, an analog circuit designed to process information, a state machine, and/or other mechanisms for electronically processing information. In some embodiments, the processors may include a plurality of processing units. These processing units may be physically located within the same device, or the processors may represent the processing functionality of a plurality of devices operating in coordination. The processors may be programmed to execute computer program instructions to perform functions described herein of subsystems described in this disclosure or other subsystems. The processors may be programmed to execute computer program instructions by software; hardware; firmware; some combination of software, hardware, or firmware; and/or other mechanisms for configuring processing capabilities on the processors.

It should be appreciated that the description of the functionality provided by the different subsystems described herein is for illustrative purposes, and is not intended to be limiting, as any of the subsystems described in this disclosure may provide more or less functionality than is described. For example, one or more of the subsystems described in this disclosure may be eliminated, and some or all of its functionality may be provided by other ones of subsystems described in this disclosure. As another example, additional subsystems may be programmed to perform some, or all of the functionality attributed herein to one of the subsystems described in this disclosure.

With respect to the components of computing devices described in this disclosure, each of these devices may receive content and data via input/output (I/O) paths. Each of these devices may also include processors and/or control circuitry to send and receive commands, requests, and other suitable data using the I/O paths. The control circuitry may comprise any suitable processing, storage, and/or I/O circuitry. Further, some or all of the computing devices described in this disclosure may include a user input interface and/or user output interface (e.g., a display) for use in receiving and displaying data. In some embodiments, a display such as a touchscreen may also act as a user input interface. It should be noted that in some embodiments, one or more devices described in this disclosure may have neither user input interface nor displays and may instead receive and display content using another device (e.g., a dedicated display device such as a computer screen and/or a dedicated input device such as a remote control, mouse, voice input, etc.). Additionally, one or more of the devices described in this disclosure may run an application (or another suitable program) that performs one or more operations described in this disclosure.

Although the present invention has been described in detail for the purpose of illustration based on what is currently considered to be the most practical and preferred embodiments, it is to be understood that such detail is solely for that purpose and that the invention is not limited to the disclosed embodiments but, on the contrary, is intended to cover modifications and equivalent arrangements that are within the scope of the appended claims. For example, it is to be understood that the present invention contemplates that, to the extent possible, one or more features of any embodiment may be combined with one or more features of any other embodiment.

As used throughout this application, the word “may” is used in a permissive sense (i.e., meaning having the potential to), rather than a mandatory sense (i.e., meaning must). The words “include,” “including,” “includes,” and the like mean including, but not limited to. As used throughout this application, the singular forms “a,” “an,” and “the” include plural referents unless the context clearly indicates otherwise. Thus, for example, reference to “an element” or “the element” includes a combination of two or more elements, notwithstanding the use of other terms and phrases for one or more elements, such as “one or more.” The term “or” is non-exclusive (i.e., encompassing both “and” and “or”), unless the context clearly indicates otherwise. Terms describing conditional relationships (e.g., “in response to X, Y,” “upon X, Y,” “if X, Y,” “when X, Y,” and the like) encompass causal relationships in which the antecedent is a necessary causal condition, the antecedent is a sufficient causal condition, or the antecedent is a contributory causal condition of the consequent (e.g., “state X occurs upon condition Y obtaining” is generic to “X occurs solely upon Y” and “X occurs upon Y and Z”). Such conditional relationships are not limited to consequences that instantly follow the antecedent obtaining, as some consequences may be delayed, and in conditional statements, antecedents are connected to their consequents (e.g., the antecedent is relevant to the likelihood of the consequent occurring). Statements in which a plurality of attributes or functions are mapped to a plurality of objects (e.g., a set of processors performing steps/operations A, B, C, and D) encompass all such attributes or functions being mapped to all such objects and subsets of the attributes or functions being mapped to subsets of the attributes or functions (e.g., both/all processors each performing steps/operations A-D, and a case in which processor 1 performs step/operation A, processor 2 performs step/operation B and part of step/operation C, and processor 3 performs part of step/operation C and step/operation D), unless otherwise indicated. Further, unless otherwise indicated, statements that one value or action is “based on” another condition or value encompass both instances in which the condition or value is the sole factor and instances in which the condition or value is one factor among a plurality of factors.

Unless the context clearly indicates otherwise, statements that “each” instance of some collection has some property should not be read to exclude cases where some otherwise identical or similar members of a larger collection do not have the property (i.e., each does not necessarily mean each and every). Limitations as to the sequence of recited steps should not be read into the claims unless explicitly specified (e.g., with explicit language like “after performing X, performing Y”) in contrast to statements that might be improperly argued to imply sequence limitations (e.g., “performing X on items, performing Y on the X'ed items”) used for purposes of making claims more readable rather than specifying a sequence. Statements referring to “at least Z of A, B, and C,” and the like (e.g., “at least Z of A, B, or C”), refer to at least Z of the listed categories (A, B, and C) and do not require at least Z units in each category. Unless the context clearly indicates otherwise, it is appreciated that throughout this specification discussions utilizing terms such as “processing,” “computing,” “calculating,” “determining,” or the like refer to actions or processes of a specific apparatus, such as a special purpose computer or a similar special purpose electronic processing/computing device. Furthermore, unless indicated otherwise, updating an item may include generating the item or modifying an existing item. Thus, updating a record may include generating a record or modifying the value of an already-generated value in a record. Additionally, as used in the specification, “a portion” refers to a part of, or the entirety of (i.e., the entire portion), a given item (e.g., data) unless the context clearly dictates otherwise.

Unless the context clearly indicates otherwise, ordinal numbers used to denote an item do not define the item's position. For example, an item that may be the first item of a set of items even if the item is not the first item to have been added to the set of items or is otherwise indicated to be listed as the first item of an ordering of the set of items. Thus, for example, if a set of items is sorted in a sequence from “item 1,” “item 2,” and “item 3,” the first item of a set of items may be “item 2” unless otherwise stated.

### Enumerated Embodiments

The present techniques will be better understood with reference to the following enumerated clauses:
1. A method comprising: obtaining, via one or more sensors, feedback data while image content is being presented by a display of a head-mounted device; and generating an eye condition indication based on the feedback data.
2. A method comprising: in connection with a first application presenting image content to a user via an inward-facing display of a head-mounted device, obtaining, via one or more sensors of the head-mounted device measuring reflections off an eye of the user, feedback data while the image content is being presented; and generating, via a second application that is sandboxed with respect to the first application, an eye condition indication based on the feedback data.
3. A method comprising: in connection with a first application presenting image content to a user via an inward-facing display of a head-mounted device, obtaining, via one or more sensors of the head-mounted device measuring reflections off an eye of the user, feedback data while the image content is being presented; providing to the feedback data to a second application that is sandboxed with respect to the first application, generating, via the second application, an eye condition indication based on the feedback data.
4. A method comprising: in connection with a presenting application presenting image content to a user of the head-mounted device via an inward-facing display, obtaining, via one or more inward-facing sensors, eye-related feedback data comprising (i) eye response data corresponding to eye responses to the presentation of the image content and (ii) reflected image content data corresponding to reflections off an eye while the image content is being presented; providing the eye-related feedback data to a sandboxed vision testing application that is sandboxed from the presenting application; generating, via the sandboxed vision testing application an eye condition indication, a sequence of reconstructed images that approximate the image content based on the reflected image content data, wherein the image content stored in memory and used by the presenting application is not accessible to the sandboxed vision testing application; generating, based on the sequence of reconstructed images, a set of expected eye measurements representing an expected response of the eye to the sequence of reconstructed images expected for a healthy eye; and generating an eye condition indication by using a machine learning model to compare the set of expected eye measurements with the eye-related feedback data.
5. A method comprising: obtaining, via one or more sensors of a head-mounted device, eye-related feedback data corresponding to (i) eye responses to a presentation of image content and (i) reflections off an eye while the image content is being presented to a user by an inward-facing display of the head-mounted device using a first application; providing the eye-related feedback data to a second application that is sandboxed from the first application; generating, by the second application, an eye condition indication by providing the eye-related feedback data to a machine learning model, wherein: the second application generates simulated input data based on the eye-related feedback data; and generating the eye condition indication comprises generating the eye condition indication based on the simulated input data.
6. A method comprising: determining a prediction model output using a first prediction model, based on feedback data stream obtained via a set of inward-facing sensors of a head-mounted device; and in response to the prediction model output, generating an eye condition indication by providing, as a set of inputs to a second prediction model, the feedback data stream.
7. A method comprising: obtaining, via one or more sensors of a head-mounted device, a feedback data stream; determining a prediction model output using a first prediction model based on the feedback data stream; and in response to the prediction model output, generating an eye condition indication by providing, as a set of inputs to a second prediction model, the feedback data stream.
8. A method comprising: in connection with a presentation of image content via an inward-facing display of a head-mounted device, obtaining, via one or more sensors of the head-mounted device, a feedback data stream while the image content is being presented; determining a prediction model output using a first prediction model based on the feedback data stream; in response to the prediction model output, generating an eye condition indication by providing, as a set of inputs to a second prediction model, the feedback data stream.
9. A method comprising: in connection with a first application presenting image content to an eye of a user, via an inward-facing display of a head-mounted device, obtaining, via one or more inward-facing sensors of the head-mounted device, a feedback data stream comprising eye response data and reflection data, while the image content is being presented during a first period; determining a binary value by providing, as learning model inputs, the feedback data stream to a screening machine learning model of a second application that is sandboxed from the first application; and in response to the binary value, generating an eye condition indication for the eye by providing, as a set of inputs to a greater-complexity machine learning model during a second period after the first period, the feedback data stream and historical data related to feedback obtained during the first period, while the first application continues to present the image content.
10. A method comprising: obtaining, via a set of inward-facing sensors of a head-mounted device, feedback data stream associated with measurements of an eye during a first period, wherein a first application presents image content to the eye during the first period via an inward-facing display of the head-mounted device; determining a prediction model output using a first prediction model of a second application that is sandboxed from the first application based on the feedback data stream; in response to the prediction model output, generating an eye condition indication by providing, as a set of inputs to a second prediction model during a second period that follows the first period, the feedback data stream and a historical data related to feedback obtained during the first period, while the first application continues to present the image content.
11. The method of any of the embodiments above, further comprising: projecting infrared light via an infrared emitting device of the head-mounted device onto an eye; and retrieving infrared light reflection data based on the infrared light reflected off the eye, wherein the feedback data stream comprises the infrared light reflection data.
12. The method of any of the embodiments above, further comprising: detecting that a visible light output of the inward-facing display is too low; and stopping a use of the first prediction model based on the detecting that the visible light output is too low.
13. The method of any of the embodiments above, wherein determining the prediction model output comprises: generating, while the image content is being presented, predicted reflection image data based on image data associated with the image content by providing the image data to the first prediction model; determining a set of differences based on the predicted reflection image data and an image of the feedback data stream; and determining the prediction model output based on the set of differences.
14. The method of any of the embodiments above, wherein providing the feedback data stream to the second prediction model comprises: determining a reflection surface area based on the feedback data stream; and determining a result, indicating whether the reflection surface area is equal to or is greater than a size threshold, wherein providing the set of inputs to the second prediction model comprises providing the feedback data stream to the second prediction model based on the result.
15. The method of any of the embodiments above, wherein providing the feedback data stream to the first prediction model comprises: determining a reflection brightness are based on the feedback data stream; determining a result, indicating whether the reflection brightness is equal to or is greater than a brightness threshold; and providing the feedback data stream to the first prediction model based on the result.
16. The method of any of the embodiments above, further comprising: retrieving, via a network connection of the head-mounted device, a data stream of the image content; and presenting the image content obtained via the data stream.
17. The method of any of the embodiments above, wherein providing the set of inputs to the second prediction model comprises selecting the second prediction model in lieu of multiple other prediction models stored in a memory of the head-mounted device based on the prediction model output.
18. The method of any of the embodiments above, wherein generating the eye condition indication comprises stopping an execution of the first prediction model.
19. The method of any of the embodiments above, wherein the prediction model output is a numeric score, and wherein stopping an execution of the first prediction model comprises: determining a result indicating whether the numeric score satisfies an activation score threshold; and stopping the execution of the first prediction model based the result.
20. The method of any of the embodiments above, wherein: the second prediction model is associated with a first set of eye conditions; a memory of the head-mounted device stores a third prediction model that is associated with a second set of eye conditions; and providing the set of inputs to the second prediction model comprises: selecting a candidate condition of the first set of eye conditions associated with the second prediction model based on the prediction model output; and selecting the second prediction model in lieu of the third prediction model based on an association between the second prediction model and the first set of eye conditions.
21. The method of any of the embodiments above, wherein the first set of eye conditions indicates at least one of a condition related to keratoconus, marginal degeneration, ectasia, or keratoglobus.
22. The method of any of the embodiments above, further comprising: receiving, via a network connection, an indication of a stimulus in stored image content that is to be presented, wherein presenting the image content comprises presenting the stored image content; determining, based on the indication, that the stimulus is to be presented within a time threshold; and providing the feedback data stream to the second prediction model independently of the prediction model output.
23. The method of any of the embodiments above, wherein generating the eye condition indication comprises generating a first eye condition indication that indicates a healthy eye, further comprising: determining a result indicating that the second prediction model indicates the first eye condition indication for a duration threshold; and stop executing the second prediction model based on the result.
24. The method of any of the embodiments above, wherein determining the prediction model output comprises: generated, while the image content is being presented, predicted reflection image data based on image data associated with the image content by providing the image data to the first prediction model; determining a set of differences based on the predicted reflection image data and an image of the feedback data stream; and determining the prediction model output based on the set of differences.
25. The method of any of the embodiments above, wherein providing the feedback data stream to the first prediction model comprises: determining a reflection surface area based on the feedback data stream; determining a result indicating whether the reflection surface area is equal to or is greater than a size threshold; and providing the feedback data stream to the first prediction model based on the result.
26. The method of any of the embodiments above, wherein providing the feedback data stream to the first prediction model comprises: determining a reflection brightness are based on the feedback data stream; determining a result indicating whether the reflection brightness is equal to or is greater than a brightness threshold; and providing the feedback data stream to the first prediction model based on the result.
27. The method of any of the embodiments above, further comprising: retrieving, via a network connection of the head-mounted device, a data stream of the image content; and presenting the image content obtained via the data stream.
28. The method of any of the embodiments above, further comprising projecting infrared light, wherein obtaining the eye-related feedback data comprises obtaining a set of infrared reflection measurements of the infrared light reflected from the eye, and wherein generating the eye condition indication comprises generating the eye condition indication based on the set of infrared reflection measurements.
29. The method of any of the embodiments above, wherein projecting the infrared light comprises projecting both the infrared light and the image content with the inward-facing display.
30. The method of any of the embodiments above, wherein projecting the infrared light comprises projecting the infrared light with a set of infrared diodes separate from the inward-facing display.
31. The method of any of the embodiments above, further comprising: obtaining, with the second application, a set of display utilization data of the head-mounted device while the image content is being projected; and determining a set of brightness values based on the set of display utilization data, generating the eye condition indication comprises generating the eye condition indication based on a difference between the set of brightness values and a measured brightness indicated by the eye-related feedback data.
32. The method of any of the embodiments above, wherein the simulated input data comprises a first set of reconstructed images, and wherein generating the eye condition indication comprises: obtaining, with the second application, a set of display utilization data of the head-mounted device while the image content is being projected; transforming the set of display utilization data into a second set of reconstructed images; and determining a set of differences between the first set of reconstructed images and the second set of reconstructed images, wherein determining the eye condition indication comprises determining the eye condition indication based on the set of differences.
33. The method of any of the embodiments above, further comprising: determining, based on the simulated input data, a set of user interface locations for visual stimuli; and generating a set of expected response values based on the set of user interface locations, wherein determining the eye condition indication comprises determining the eye condition indication based on the set of expected response values.
34. The method of claim 8, wherein: determining the set of user interface locations comprises determining the set of user interface locations based on a sequence of frames of the simulated input data while the image content is being presented by the head-mounted device; using the machine learning model to generate a predicted eye response value for a gaze change to a new location of the set of user interface locations, wherein the set of user interface locations comprises the predicted eye response value; determining a reaction difference between the predicted eye response value and an eye response value indicated by the eye-related feedback data; and generating the eye condition indication based on the reaction difference.
35. The method of any of the embodiments above, wherein the eye condition indication indicates a dry eye condition.
36. The method of any of the embodiments above, wherein the eye condition indication indicates a visual field that is less than 110 degrees in a temporal direction, less than 60 degrees in a nasal direction, less than 60 degrees in a superior direction, or less than 75 degrees in an inferior direction.
37. The method of any of the embodiments above, further comprising: detecting, via orientation sensors of the head-mounted device, a measured head shift time that indicates a shift from a first head orientation to a second head orientation while the image content is being presented; and generating a predicted head shift time based on the feedback data, wherein generating the eye condition indication comprises generating the eye condition indication based on the predicted head shift time and the measured head shift time.
38. The method of any of the embodiments above, wherein the eye condition indication is related to at least one of keratoconus, marginal degeneration, ectasia, keratoglobus, or other eye condition.
39. The method of any of the embodiments above, further comprising projecting infrared light, wherein obtaining the feedback data comprises obtaining a set of infrared reflection measurements of the infrared light reflected from the eye, and wherein generating the eye condition indication comprises generating the eye condition indication based on the set of infrared reflection measurements.
40. The method of any of the embodiments above, wherein generating the eye condition indication comprises: obtaining a set of display utilization data of the head-mounted device while the image content is being projected; determining a set of brightness values based on the set of display utilization data; and generating a set of expected response values based on the set of brightness values, wherein generating the eye condition indication comprises generating the eye condition indication based on a difference between an expected reflected brightness indicated by the set of expected response values and a measured brightness indicated by the feedback data.
41. The method of any of the embodiments above, wherein the feedback data comprises a first set of reconstructed images, and wherein generating the eye condition indication comprises: obtaining, with the second application, a set of display utilization data of the head-mounted device while the image content is being projected; transforming the set of display utilization data into a second set of reconstructed images; determining a set of differences between the first set of reconstructed images and the second set of reconstructed images, wherein determining the eye condition indication comprises determining the eye condition indication based on the set of differences.
42. The method of any of the embodiments above, wherein determining the prediction model output comprises: generated, while the image content is being presented, predicted pupil reaction data or predicted eye movement data based on image data associated with the image content by providing the image data to the first prediction model; determining a set of differences based on the predicted pupil reaction data or the predicted eye movement data and measured eye-related data of the feedback data stream; and determining the prediction model output based on the set of differences.
43. The method of any of the embodiments above, wherein: the second application generates simulated input data based on the feedback data; generating the eye condition indication comprises generating the eye condition indication based on the simulated input data; generating the eye condition indication comprises: determining, based on the simulated input data, a set of user interface locations for visual stimuli; and generating a set of expected response values based on the set of user interface locations, wherein determining the eye condition indication comprises determining the eye condition indication based on the set of expected response values.
44. The method of any of the embodiments above, wherein generating the eye condition indication comprises: determining an expected eye response using the machine learning model; and providing the feedback data and the expected eye response to a rules-based system to generate the eye condition indication.
45. A tangible, non-transitory, machine-readable medium storing instructions that, when executed by a data processing apparatus, cause the data processing apparatus to perform operations comprising those of any of embodiments 1-44.
46. A system comprising one or more processors; and memory storing instructions that, when executed by the processors, cause the processors to effectuate operations comprising those of any of embodiments 1-44.
47. A system comprising means for performing any of embodiments 1-44.
48. A head-mounted device comprising: one or more inward-facing sensors and an inward-facing display; one or more processors and non-transitory media storing instructions that, when executed by the one or more of processors, cause the processors to effectuate operations comprising those of any of embodiments 1-44.

What is claimed is:

1. A head-mounted device for passive eye condition detection via a sandboxed application during content presentation independent of the sandboxed application, the head-mounted device comprising:
one or more inward-facing sensors and an inward-facing display;
one or more processors and non-transitory media storing instructions that, when executed by the one or more processors, perform operations comprising:
in connection with a presenting application presenting image content to a user of the head-mounted device via the inward-facing display, obtaining, via the one or more inward-facing sensors, eye-related feedback data comprising (i) eye response data corresponding to eye responses to the presentation of the image content and (ii) reflected image content data corresponding to reflections off an eye while the image content is being presented;
providing the eye-related feedback data to a sandboxed vision testing application that is sandboxed from the presenting application;
generating, via the sandboxed vision testing application, a sequence of reconstructed images that approximate the image content based on the reflected image content data, wherein the image content stored in memory and used by the presenting application is not accessible to the sandboxed vision testing application;
generating, based on the sequence of reconstructed images, a set of expected eye measurements representing an expected response of the eye to the sequence of reconstructed images expected for a healthy eye; and
generating an eye condition indication by using a machine learning model to compare the set of expected eye measurements with the eye-related feedback data.

2. A method comprising:
obtaining, via one or more sensors of a head-mounted device, eye-related feedback data corresponding to (i) eye responses to a presentation of image content and (i) reflections off an eye while the image content is being presented to a user by an inward-facing display of the head-mounted device using a first application;
providing the eye-related feedback data to a second application that is sandboxed from the first application;
generating, by the second application, an eye condition indication by providing the eye-related feedback data to a machine learning model, wherein:
the second application generates simulated input data based on the eye-related feedback data; and
generating the eye condition indication comprises generating the eye condition indication based on the simulated input data.

3. The method of claim 2, further comprising projecting infrared light, wherein obtaining the eye-related feedback data comprises obtaining a set of infrared reflection measurements of the infrared light reflected from the eye, and wherein generating the eye condition indication comprises generating the eye condition indication based on the set of infrared reflection measurements.

4. The method of claim 3, wherein projecting the infrared light comprises projecting both the infrared light and the image content with the inward-facing display.

5. The method of claim 3, wherein projecting the infrared light comprises projecting the infrared light with a set of infrared diodes separate from the inward-facing display.

6. The method of claim 2, further comprising:
obtaining, with the second application, a set of display utilization data of the head-mounted device while the image content is being projected; and
determining a set of brightness values based on the set of display utilization data, generating the eye condition indication comprises generating the eye condition indication based on a difference between the set of brightness values and a measured brightness indicated by the eye-related feedback data.

7. The method of claim 2, wherein the simulated input data comprises a first set of reconstructed images, and wherein generating the eye condition indication comprises:
obtaining, with the second application, a set of display utilization data of the head-mounted device while the image content is being projected;
transforming the set of display utilization data into a second set of reconstructed images; and
determining a set of differences between the first set of reconstructed images and the second set of reconstructed images, wherein determining the eye condition indication comprises determining the eye condition indication based on the set of differences.

8. The method of claim 2, further comprising:
determining, based on the simulated input data, a set of user interface locations for visual stimuli; and
generating a set of expected response values based on the set of user interface locations, wherein determining the eye condition indication comprises determining the eye condition indication based on the set of expected response values.

9. The method of claim 8, wherein:
determining the set of user interface locations comprises determining the set of user interface locations based on a sequence of frames of the simulated input data while the image content is being presented by the head-mounted device;
using the machine learning model to generate a predicted eye response value for a gaze change to a new location of the set of user interface locations, wherein the set of user interface locations comprises the predicted eye response value;
determining a reaction difference between the predicted eye response value and an eye response value indicated by the eye-related feedback data; and
generating the eye condition indication based on the reaction difference.

10. The method of claim 2, wherein the eye condition indication indicates a dry eye condition.

11. The method of claim 2, wherein the eye condition indication indicates a visual field that is less than 110 degrees in a temporal direction, less than 60 degrees in a nasal direction, less than 60 degrees in a superior direction, or less than 75 degrees in an inferior direction.

12. One or more non-transitory machine-readable media storing program instructions that, when executed by one or more processors, causes the one or more processors to perform operations comprising:
in connection with a first application presenting image content to a user via an inward-facing display of a head-mounted device, obtaining, via one or more sensors of the head-mounted device measuring reflections off an eye of the user, feedback data while the image content is being presented;
providing to the feedback data to a second application that is sandboxed with respect to the first application, generating, via the second application, an eye condition indication by using a machine learning model based on the feedback data.

13. The one or more machine-readable media of claim 12, the operations further comprising:
detecting, via orientation sensors of the head-mounted device, a measured head shift time that indicates a shift from a first head orientation to a second head orientation while the image content is being presented; and
generating a predicted head shift time based on the feedback data, wherein generating the eye condition indication comprises generating the eye condition indication based on the predicted head shift time and the measured head shift time.

14. The one or more machine-readable media of claim 12, wherein the eye condition indication indicates at least one of a condition related to keratoconus, marginal degeneration, ectasia, or keratoglobus.

15. The one or more machine-readable media of claim 12, the operations further comprising projecting infrared light, wherein obtaining the feedback data comprises obtaining a set of infrared reflection measurements of the infrared light reflected from the eye, and wherein generating the eye condition indication comprises generating the eye condition indication based on the set of infrared reflection measurements.

16. The one or more machine-readable media of claim 12, wherein generating the eye condition indication comprises:
obtaining a set of display utilization data of the head-mounted device while the image content is being projected;
determining a set of brightness values based on the set of display utilization data; and
generating a set of expected response values based on the set of brightness values, wherein generating the eye condition indication comprises generating the eye condition indication based on a difference between an expected reflected brightness indicated by the set of expected response values and a measured brightness indicated by the feedback data.

17. The one or more machine-readable media of claim 12, wherein the feedback data comprises a first set of reconstructed images, and wherein generating the eye condition indication comprises:
obtaining, with the second application, a set of display utilization data of the head-mounted device while the image content is being projected;
transforming the set of display utilization data into a second set of reconstructed images;
determining a set of differences between the first set of reconstructed images and the second set of reconstructed images, wherein determining the eye condition indication comprises determining the eye condition indication based on the set of differences.

18. The one or more machine-readable media of claim 12, wherein:
the second application generates simulated input data based on the feedback data;
generating the eye condition indication comprises generating the eye condition indication based on the simulated input data;
generating the eye condition indication comprises:
determining, based on the simulated input data, a set of user interface locations for visual stimuli; and
generating a set of expected response values based on the set of user interface locations, wherein determining the eye condition indication comprises determining the eye condition indication based on the set of expected response values.

19. The one or more machine-readable media of claim 12, wherein generating the eye condition indication comprises:
determining an expected eye response using the machine learning model; and
providing the feedback data and the expected eye response to a rules-based system to generate the eye condition indication.

20. The one or more machine-readable media of claim 12, wherein the eye condition indication indicates a visual field that is less than 110 degrees in a temporal direction, less than 60 degrees in a nasal direction, less than 60 degrees in a superior direction, or less than 75 degrees in an inferior direction.
